(19) United States
(12) Patent Application Publication
(10) Pub. No.: US 2013/0231394 A1
(43) Pub. Date: Sep. 5, 2013

(54) ANTIMICROBIAL WOUND DRESSING

(71) Applicants: Andreas Arndt, Luzern (CH); Andrea Preuss, Olten (CH); Jürgen Schmitt, Kirchhain (DE); Martin Sippel, Melsungen (DE); Thomas Riemann, Grossalmerode (DE); André Weiss, Guxhagen (DE); Axel Kramer, Greifswald (DE); Gerald Mueller, Hinrichshagen (DE)

(72) Inventors: Andreas Arndt, Luzern (CH); Andrea Preuss, Olten (CH); Jürgen Schmitt, Kirchhain (DE); Martin Sippel, Melsungen (DE); Thomas Riemann, Grossalmerode (DE); André Weiss, Guxhagen (DE); Axel Kramer, Greifswald (DE); Gerald Mueller, Hinrichshagen (DE)

(73) Assignee: B. Braun Melsungen AG, Melsungen (DE)

(21) Appl. No.: 13/637,111

(22) PCT Filed: Mar. 25, 2011
(86) PCT No.: PCT/EP11/54617

§ 371 Date: Nov. 27, 2012

(30) Foreign Application Priority Data
Mar. 26, 2010 (DE) 10-2010013075.3

(51) Int. Cl. A61K 31/155

(52) U.S. Cl. CPC A61K 31/155
USPC 514/635

(57) ABSTRACT

The invention relates to a wound dressing comprising a polymer substrate and a compound comprising a) at least one antimicrobial active agent and b) an agent that reduces the cytotoxicity, comprising an oil-in-water emulsion that additionally has one or more alkanediol(s) and/or one or more glyceryl ether(s).

The invention relates to a wound dressing comprising a polymeric substrate and a composition comprising at least one antimicrobially active substance and a cytotoxicity-reducing agent. In addition, the invention relates to the preparation of such a wound dressing.

Antimicrobially active substances are used in different cosmetic and medicinal products both for preservation of the respective product and for achieving an antimicrobial effect on the skin or mucosa, i.e., for disinfection, antiseptic treatment and reduction of an infection risk. Because of their intended biological effect, antimicrobially active substances always bear some risk of damage to human cells. In particular, it is desirable for such active substances to show a non-specific mechanism of action in order to prevent any development of a resistance or even cross-resistance against antibiotics.

On the other hand, products containing such active substances should not have a damaging effect on human tissue. This is of even greater importance if such products come into contact, not only with healthy skin, but especially for products employed in wound treatment.

Polymeric guanidines as well as polymeric biguanides, especially polyhexamethylene biguanide, are antimicrobially active substances that are employed mainly for the preservation of cosmetic formulations, but also increasingly in wound treatment, for example, in wound antiseptics, wound irrigation solutions, and wound dressings.

Especially in the field of wound treatment, i.e., also for objects that come into contact with wounds, it is of critical importance that the cytotoxic effect is kept as low as possible. In the prior art, different strategies of how an antimicrobial effect can be achieved on the one hand and a cytotoxic effect can be prevented at the same time have become established.

EP-A1-1 175 148 discloses polymeric wound dressings made of polyurethane foams, in the structure of which the antimicrobially active substance, for example, the polyhexamethylene biguanide (PHMB), is chemically covalently bonded to the polymer structure. Because of this covalent bonding, there is a limited bioavailability and thus a low risk of cytotoxic effects, although the antimicrobial effectiveness is reduced at the same time.

WO-A2-2007/089763 describes super-soft foam materials for use as wound dressings that contain one or more antimicrobially active substances. These active substances are employed in a suitable amount, so that they still show an acceptable efficiency without having a cytotoxic effect. The balance between the desirable antimicrobial and the undesirable cytotoxic effectiveness is sought exclusively through the selection of a suitable concentration of the antimicrobially active substance.

EP-A1-1 830 869 describes a cellular hydrophilic polyurethane containing polyhexamethylene biguanide or its hydrochloride as an antiseptically active substance, wherein said antiseptically active agent is in microparticulate distribution and/or homogeneous solution, and said antiseptic agent is contained in the polyurethane gel together with a superabsorber. The mode of action is mainly based on the fact that the germ-containing wound secretion is taken up into the wound dressing and never released again, whereby an enhancement of effectiveness is achieved with very low concentrations of the antimicrobial agent and apparently a very low release.

WO-A1-2009/010068 discloses a wound dressing consisting of a polyurethane foam, the foam containing an antimicrobially active substance that can be released from the foam by means of water and at the same time serves as a modulator of the foam flexibility. A way to avoid possible undesirable side effects is not shown here.

DE-A1-10 2007 030 931 describes compositions for the external treatment of wounds, the compositions containing a nutritive substance, a disinfecting substance and a protease-inhibiting substance. However, a method for reducing a possible cytotoxic effect from disinfecting ingredients, such as polyhexanide, is not described.

Especially in the use of wound dressings that can release the antimicrobially active substance, especially active substances based on guanidine derivatives or biguanide derivatives, it is the object of the present invention to provide a wound dressing that has an antimicrobial effect on the one hand and at the same time exerts as low as possible a cytotoxic effect on the other.

Surprisingly, it has been found that the problems stated in the prior art can be solved by a wound dressing containing a substrate and a composition with at least one antimicrobially active substance and at least one cytotoxicity-reducing agent.

The present invention relates to a wound dressing comprising a polymeric substrate and a composition comprising
a) at least one antimicrobially active substance; and
b) a cytotoxicity-reducing agent comprising an oil-in-water emulsion that additionally contains one or more alkanediols and/or one or more glycerol ethers.

The polymeric substrate of the wound dressing according to the invention is preferably designed to absorb liquids. Preferably, the polymeric substrate is a cellular foam, for example, with a sponge-like structure.

The selection of the polymeric substrate is not limited as long as it is suitable for contact with skin and wounds and, in particular, does not provoke any damaging effect. Polymeric substrates of natural and artificial origin are basically suitable. More preferably, the polymeric substrate is selected from the group consisting of polyurethanes, polyethers, cellulose materials, cellulose derivatives, cotton, rayon, polyester, polyvinyl alcohol, polyvinyl acetate, polysulfones, polyacrylates, polyolefins, polyamides, alginates, chitosan, and any mixtures and combinations thereof.

Among the polyesters, polyethylene terephthalate has proven particularly suitable. Among the polyolefins, both polyethylenes, polypropylenes and any copolymers thereof may be employed. Suitable polyamides include, for example, nylon, especially nylon 6,6 (the polycondensation product of 1,6-diaminohexane with adipic acid), or nylon 6, which is referred to as polycaprolactam. According to the present invention, the polymeric substrate is more preferably a polyurethane obtainable by reacting a component having at least one isocyanate group, preferably two isocyanate groups, with at least one polyol, preferably a polyether polyol and/or a polyester polyol.

Suitable polymeric substrates for use in wound dressings are described, for example, in WO 2009/010068 A1 and WO 2007/089763 A2.

A crosslinked hydrophilic polyurethane that is absorbent is particularly preferred.

Polymeric substrates in the form of a polyurethane foam are particularly suitable. For this purpose, the polyurethane foams can be prepared by NCO-terminated prepolymers in combination with the composition, preferably aqueous composition, to be employed according to the invention. The NCO-terminated prepolymer polymerizes quickly in an aqueous phase, for example one containing surfactants, to form a foam. The foam is preferably fabricated in the shape of a wound dressing already during the preparation thereof.

According to the present invention, NCO-terminated polyether prepolymers are particularly suitable for the preparation of polyurethane foams. Such polyether prepolymers include hydrophilic polyether polyols, in particular. Suitable hydrophilic polyether polyols include the reaction products of ethylene oxide or combinations of ethylene oxide with other alkylene oxides, and one or more components having at least two active hydrogen atoms, such as polyols, polyphenols, polyamines, polycarboxylic acids, phosphoric acids, and the like. Examples of suitable polyols include, for example, ethylene glycol, propylene glycol, 1,3- and 1,4-butanediols, 1,6-hexanediol, diethylene glycol, bis(hydroxymethyl)cyclohexane, bis(hydroxyethyl)benzene, hydrogenated bisphenol A, hydrogenated bisphenol F, polytetramethylene glycols, polyesterdiols and silanol-terminated polysiloxanes, glycerol, trimethylolpropane, trimethylolethane, 1,2,3-butanetriol, 1,2,6-hexanetriol, polyestertriols, pentaerythritol, diglycerol, α-methylglucosides, sorbitol, xylitol, mannitol, glucose, fructose, sucrose, and the like. Examples of suitable phenols include hydroquinone, catechol, resorcinol, pyrogallol, and bisphenols, such as bisphenol A, bisphenol F, bisphenol S, and the like. In addition, ethanolamines as well as aliphatic, aromatic, alicyclic and araliphatic polyamines, such as C₂-C₆ alkylenediamines, diethylenetriamines, toluenediamines, phenylenediamines, xylylenediamines, methylenediamines, diphenyletherdiamines, isophoronediamines, cyclohexylenediamines, cyclohexylmethanediamines, and the like may be used.

Suitable alkylene oxides that may be employed in combination with ethylene oxide for preparing the polyether polyols include, for example, propylene oxide, 1,2-, 2,3-, 1,3- and 1,4-butylene oxide, styrene oxide, epichlorohydrin, and the like. The addition of the ethylene oxide or of the combination of ethylene oxide with other alkylene oxides to the compounds having active hydrogen can be effected in methods familiar to the skilled person with or without catalysts. The addition of the ethylene oxides and alkylene oxides may be effected randomly to form mixed polyethers, or in a block mode to form block polymers.

In a preferred embodiment, the polyols for preparing the NCO-terminated prepolymers have an oxyethylene content of at least 30% by weight, more preferably at least 50% by weight, and especially at least 90% by weight, and a mean hydroxy group number of from 2 to 8, especially from 2 to 4.

The above described polyether polyols are subsequently capped with isocyanates, for example, aromatic isocyanates or aliphatic isocyanates. Suitable aromatic isocyanates include those having from 6 to 20 carbon atoms (excluding the carbon atoms of the NCO group). Suitable examples include p-phenylene diisocyanate (PDI), 4,4′-diphenylmethane diisocyanate (MDI), and positional isomers thereof, 2,4- and/or 2,6-toluene diisocyanate (TDI), and positional isomers thereof, 3,4-dichlorophenyl diisocyanate, dicyclohexylmethane 4,4′-diisocyanate (HMDI), 1,6-hexamethylene diisocyanate (HDI), and positional isomers thereof, and the like. Suitable aliphatic isocyanates include isophorone diisocyanate (IPDI) and the like.

For the preparation of the NCO-terminated hydrophilic urethane prepolymers, the isocyanates are reacted with at least one hydrophilic polyether polyol, preferably in such a ratio that the ratio of NCO/OH is from 1.5 to 5.0, more preferably from 1.7 to 3.0. The reaction of the isocyanates with the polyether polyols to form the prepolymers may be performed by methods familiar to the skilled person. In preferred embodiments, the reaction may also be performed in the presence of catalysts.

The NCO content of the NCO-terminated hydrophilic prepolymers is preferably from 1 to 10% by weight, more preferably from 2 to 8% by weight. Suitable NCO-terminated polyether prepolymers that may be used for the preparation of the wound dressing according to the present invention are disclosed, for example, in documents U.S. Pat. No. 3,903,232 and U.S. Pat. No. 4,137,200. Such prepolymers have an average isocyanate functionality of, for example, above 2, in some embodiments even a functionality of 2 to 10. Suitable “NCO-terminated polyether prepolymers” also include those commercially available under the trade mark name Hypol, such as Hypol 2000, Hypol 2002, Hypol 3000, Hypol 4000, Hypol 5000, Hypol X 6100, and Hypol Hydrogel. Preferred NCO-terminated polyether prepolymers have an equivalent weight (molecular weight per NCO group) of from 100 to 1000 daltons, preferably from 500 to 750 daltons.

In a particularly preferred embodiment, the prepolymers for the preparation of the polyurethane foam are combined with a previously provided aqueous composition containing at least one antimicrobially active substance and at least one or more cytotoxicity-reducing agents, to form the polyurethane foam.

In a preferred embodiment, the polymeric substrate is selected from the group consisting of polyethylene terephthalate, polyethylene, polypropylene, nylon-6,6, polycaprolactam, polyurethane, and any mixtures and combinations thereof.

In a preferred embodiment, the polymeric substrate is a polyurethane, preferably a polyurethane foamed at a temperature of from 20 to 45° C., or a thermoplastic polyurethane.

Another essential component of the wound dressing according to the invention is a composition containing at least one antimicrobially active substance and at least one cytotoxicity-reducing agent. Preferably, this composition is present during the polymerization process for preparing the polymeric substrate and is thereby incorporated into the polymer matrix. Alternatively, the composition may be applied to the polymeric substrate, for example, by immersion or spraying. If the polymeric substrate should have a gel-like consistency, the composition can be incorporated into the substrate by methods familiar to the skilled person. Preferably, the composition is liquid at 20° C. More preferably, the composition is an aqueous formulation. The pH of the composition is preferably pH 4 to 8, especially 5 to 7.

The composition comprises an antimicrobially active substance as an essential component.

The antimicrobially active substance is preferably selected from the group of components having at least one guanide and/or biguanide group, iodophors, triclosan, octenidine, quaternary ammonium compounds, lactoferrin, undecylenic acid and/or salts thereof and/or any mixtures thereof. More preferably, the antimicrobially active substance is selected from the group consisting of polymethylene biguanide, polyhexamethylene biguanide, polyhexamethylene guanide, chlorhexidine, hexetidine, iodophors, PVP-iodine, 2,4,4′-trichloro-2-hydroxydiphenyl ether, octenidine, and salts thereof, preferably hydrohalides, acetates, gluconates or sulfates thereof, especially chlorhexidine acetate, chlorhexidine gluconate, chlorhexidine sulfate, chlorhexidine hydrochloride, and octenidine hydrochloride.

More preferably, the antimicrobially active substance is an aliphatic component having one or more guanide and/or biguanide groups. According to the present invention, “aliphatic component” means that the component has no aromatic ring system.

According to the present invention, “aliphatic” also includes cycloaliphatic or heterocyclic systems as long as they have saturated ring systems.

According to the present invention, the “component having a guanide group” is a component that has the following structural element in its chemical structure:

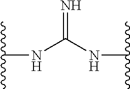 [SMILES: *NC(=N)N*]

Hereinafter, these components are referred to as “guanide” or “guanides” for the sake of simplicity.

“Components having a biguanide group” are those chemical compounds that have the following structural element:

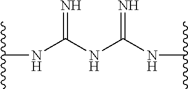 [SMILES: *NC(=N)NC(=N)N*]

Hereinafter, these components are referred to as “biguanide” or “biguanides” for the sake of simplicity.

Polyhexamethylene biguanide and/or a salt of polyhexamethylene biguanide, preferably a hydrohalide, for example, hydrochloride, has proven a particularly suitable antimicrobially active substance.

Surprisingly, it has also been found that antimicrobially active substances obtainable by the polycondensation of a guanidine acid addition salt with a mixture of amines which contains at least one diamine and/or triamine, wherein preferably at least one amine is selected from the group consisting of

i) a diamine having at least one cycloaliphatic radical; and
ii) a dialkylene triamine;
are particularly suitable.

For example, the polycondensation may be performed at temperatures of from 100° C. to 180° C., preferably from 130° C. to 160° C., for a period of preferably 30 minutes to 6 hours.

The thus obtainable polymeric or oligomeric antimicrobially active substances may be either homopolymers or copolymers. It is advantageous if the guanidine acid addition salt is guanidine hydrochloride. However, other guanidine acid addition salts based on inorganic or organic acids are also suitable, for example, the hydroxides, hydrogensulfates and acetates.

These antimicrobially active substances are characterized by particularly high biocidal properties and are therefore particularly suitable for the wound dressings according to the invention.

Preferably, the antimicrobially active substances are obtainable by the polycondensation of a guanidine acid addition salt with a mixture of amines containing at least one diamine and/or triamine, wherein at least one amine is selected from the group consisting of:

i) a diamine having at least one cycloaliphatic radical; and
ii) a dialkylene triamine.

The polymeric or oligomeric active substances obtainable by the polycondensation preferably have a polyguanidine structure or a polyiminoimidazole structure, especially when dialkylene triamines, for example, diethylene triamine, are employed.

In a preferred embodiment of the present invention, the mixture of amines comprises component i) (diamine having at least one cycloaliphatic radical) and/or component ii) (dialkylene triamine) in an amount of at least 10 mole percent, preferably at least 25 mole percent, more preferably at least 45 mole percent, especially at least 85 mole percent, specifically at least 95 mole percent, respectively based on the total mixture of amines.

Preferably, the mixture of amines additionally comprises an alkylene diamine, which is more preferably a compound of the general formula

NH₂(CH₂)ₙNH₂,

in which n is an integer of from 2 to 10, preferably 4 or 6. Preferably employed alkylene diamines have terminal amino groups. Hexamethylene diamine (hexane 1,6-diamine) is specifically preferred. The alkylene diamine can be employed in the polycondensation reaction in admixture with other diamines or triamines, wherein at least one amine is selected from the group consisting of
i) a diamine having at least one cycloaliphatic radical; and
ii) a dialkylene triamine;
preferably selected from the group consisting of 4,4′-methylenebis(cyclohexylamine) and/or diethylene triamine, to form copolymers.

Preferably, the mixture of amines may further comprise oxyalkylene diamines.

Oxyalkylene diamines having terminal amino groups are particularly suitable as oxyalkylene diamines. A preferred oxyalkylene diamine is a compound of general formula

H₂[(CH₂)₂O)]ₙ(CH₂)₂NH₂,

in which n is an integer of from 2 to 6, preferably from 2 to 5, more preferably from 2 to 4, especially 2. Polyoxyethylene diamines, especially triethylene glycol diamine, are preferred. Polyoxypropylene diamines, especially di- or tripropylene glycol diamine, may further be preferably employed.

In a preferred embodiment, the polymeric or oligomeric active substance is a homopolymer. In such cases, the mixture of amines consists of a diamine having at least one cycloaliphatic radical, or of a dialkylene triamine.

In another preferred embodiment, the mixture of amines consists of the triamine diethylene triamine. In this variant, the polymeric or oligomeric active substance is thus a homopolymer, for example, polyiminoimidazolidine.

In another preferred embodiment, the mixture of amines consists of the diamine 4,4′-methylenebis(cyclohexylamine). The homopolymer poly(4,4′-methylenebis(cyclohexylamine hydrochloride), for example, is produced therefrom by polycondensation with a guanidine acid addition salt.

The polymeric or oligomeric active substances obtainable by the polycondensation of a guanidine acid addition salt with a mixture of amines which contains at least one diamine having at least one cycloaliphatic radical are particularly preferred. Diamines having at least one cycloaliphatic radical include, for example, cycloaliphatic diamines, for example, cyclohexane diamine, cyclopentane diamine and derivatives thereof. Those diamines in which at least one NH₂ group is directly linked with the cycloaliphatic radical are particularly preferred. Those diamines in which both NH₂ groups are linked directly each with one and the same cycloaliphatic radical or with different cycloaliphatic radicals are particularly preferred. In a particular embodiment, the mixture of amines comprises 4,4′-methylenebis(cyclohexylamine).

In another embodiment of the present invention, the mixture of amines comprises at least one dialkylene triamine. The dialkylene triamines may have alkylene residues of different chain lengths. However, dialkylene triamines in which the alkylene groups have the same length are preferred. Preferred alkylene radicals include ethylene, propylene and butylene as well as hexylene. In a particularly preferred embodiment, the mixture of amines comprises the triamine diethylene triamine.

In another preferred embodiment, polymeric or oligomeric active substances used according to the invention are in the form of copolymers. These may be either randomly mixed or in the form of block copolymers. In the case of copolymers, the mixture of amines contains at least two different amines. The mixture of amines contains a first component and at least one second component, wherein preferably
said first component is a diamine or triamine selected from the group consisting of
   i) a diamine having at least one cycloaliphatic radical; and
   ii) a dialkylene triamine; and wherein
said second component is a diamine or triamine selected from the group consisting of
   i) a diamine having at least one cycloaliphatic radical;
   ii) a dialkylene triamine;
   iii) an alkylene diamine; and
   iv) an oxyalkylenediamine; and
      wherein said first component is different from said second component.

Those in which the first component is 4,4′-methylenebis(cyclohexylamine) and the second component is selected from diethylene triamine, hexamethylene diamine and triethylene glycol diamine have proven to be particularly suitable copolymeric or cooligomeric active substances.

In another preferred embodiment, the copolymeric guanidine derivative contains diethylene triamine as the first component, and the second component is selected from the group consisting of hexamethylene diamine and triethylene glycol diamine.

The wound dressing according to the invention more preferably contains a composition containing an antimicrobially active substance having a n average molecular weight within a range of from 500 to 7000, especially from 1000 to 5000, daltons.

According to the present invention, the term “polymeric guanidine derivative or biguanide” is used for guanidine derivatives or biguanides in which 2 or more repeating units are present. Thus, the term “polymer” also comprises dimers, trimers or, for example, oligomers.

Another class of polymeric guanidine derivatives is described, for example, in WO-A1-01/85676, and in WO-A1-06/047800.

Preferred antimicrobially active substances are obtainable by the polycondensation of a guanidine acid addition salt with a mixture of amines which contains at least one diamine selected from the group consisting of alkylene diamine and oxyalkylene diamine.

Preferably, the mixture of amines comprises an alkylene diamine, which is more preferably a compound of general formula

NH₂(CH₂)ₙNH₂,

in which n is an integer of from 2 to 10, preferably 4 or 6. Preferably employed alkylene diamines have terminal amino groups. Hexamethylene diamine (hexane 1,6-diamine) is specifically preferred. The alkylene diamine can be employed in the polycondensation reaction in admixture with other polyamines, for example, di- and/or triamines, to form copolymers.

Preferably, the mixture of amines comprises at least one oxyalkylene diamine.

Oxyalkylene diamines having terminal amino groups are particularly suitable as oxyalkylene diamines. A preferred oxyalkylene diamine is a compound of general formula

NH₂[CH₂)₂O)]ₙ(CH₂)₂NH₂,

in which n is an integer of from 2 to 6, preferably from 2 to 5, more preferably from 2 to 4, especially 2. Oxyethylene diamines, especially diethylene glycol diamine or triethylene glycol diamine, are preferred. Polyoxypropylene diamines, especially di- or tripropylene glycol diamine, may further be preferably employed.

Preferably, the polymeric guanidine derivative is a homopolymer. In such cases, the mixture of amines consists of the alkylene diamine or of an oxyalkylene diamine.

In another preferred embodiment, the mixture of amines consists of the alkylene diamine hexamethylene diamine (hexane 1,6-diamine). In this variant, the polymeric guanidine derivative thus consists of a homopolymer, for example, poly(hexamethylene guanidine hydrochloride) (PHMG).

In another preferred embodiment, the mixture of amines consists of the oxyalkylene diamine triethylene glycol diamine. Polycondensation thereof with a guanidine acid addition salt forms, for example, the homopolymer poly[(2-(2-ethoxy)ethoxyethyl)guanidine hydrochloride].

In another preferred embodiment, the polymeric guanidine derivatives used according to the invention are in the form of copolymers. These may be either randomly mixed or in the form of block copolymers. In the case of copolymers, the mixture of amines contains at least two different amines. The mixture of amines contains a first component and at least one second component, wherein
said first component is a diamine selected from the group consisting of an alkylene diamine and an oxyalkylenediamine; and wherein
said second component is a diamine selected from the group consisting of an alkylene diamine and an oxyalkylenediamine; and
wherein said first component is different from said second component.

Those in which the first component is alkylene diamine and the second component is an oxyalkylene diamine have proven to be particularly suitable copolymeric guanidine derivatives. Copolymeric guanidine derivatives in which the first component is hexamethylene diamine and the second component is triethylene glycol diamine in the mixture of amines are particularly preferred.

In the preparation of copolymers, the mixing ratio of the amines to be employed can be varied widely. However, preferred are copolymeric guanidine derivatives in which the monomers of the mixture of amines, i.e., the first component and the second component, are in a molar ratio of from 4:1 to 1:4, preferably from 2:1 to 1:2.

The polymeric guanidine derivatives to be employed according to the invention preferably have an average molecular weight (weight average) within a range of from 500 to 7000, especially from 1000 to 5000, daltons.

In another preferred embodiment of the present invention, the polymeric guanidine derivative to be employed according to the invention is a mixture of at least 2 different polymeric guanidine derivatives. In a specific embodiment, the mixture of the polymeric guanidine derivatives comprises both a first homopolymer based on an alkylene diamine, preferably poly(hexamethylene guanidine hydrochloride) and a second homopolymer based on an oxyalkylene diamine, for example, poly[(2-(2-ethoxy)ethoxyethyl)guanidine hydrochloride].

In a preferred embodiment, the polymeric guanidine derivative comprises the first homopolymer and the second homopolymer in a weight ratio of from 5:1 to 1:5, preferably from 1:1 to 1:4, especially from 1:2 to 1:4. In a particularly preferred embodiment, the polymeric guanidine mixture comprises poly(hexamethylene guanidine hydrochloride) (first homopolymer) and poly[(2-(2-ethoxy)ethoxyethyl)guanidine hydrochloride] (second homopolymer) in a weight ratio (of first homopolymer to second homopolymer) of from 1:1 to 1:5, preferably from 1:2 to 1:4, especially 1:3. Such mixtures are particularly suitable for the wound dressing of the present invention.

The polymeric guanidine derivatives and biguanides used according to the invention may generally be either homopolymers or copolymers. It is advantageous if the guanidine acid addition salt is guanidine hydrochloride. However, other guanidine acid addition salts based on inorganic or organic acids are also suitable, for example, the hydroxides, hydrogensulfates and acetates. Particularly suitable and effective polymeric guanidine derivatives are in the form of their hydroxide salts. These may be obtained from the corresponding chloride salts, for example, by anion exchange.

In addition, the composition contains a cytotoxicity-reducing agent comprising an oil-in-water emulsion that additionally contains one or more alkanediols and/or one or more glycerol ethers.

Preferably, the composition additionally contains further cytotoxicity-reducing agents selected from the group consisting of
i) one or more betaine derivatives;
ii) one or more fatty alcohol alkoxylates;
iii) one or more glycerol esters;
iv) allantoin; and
v) panthenol and/or pantothenic acid.

Components i) to v) may each be present individually or in any combinations in the composition.

An essential component of the composition comprises an oil-in-water emulsion.

Surprisingly, it has been found that oil-in-water emulsions can significantly reduce or completely suppress the cytotoxicity of the antimicrobially active substances employed. In a preferred embodiment, the oil-in-water emulsion comprises at least one phospholipid and at least one oil component.

In principle, any oil components are suitable for the oil-in-water emulsions, but oil components of natural origin are preferred. Particularly preferred oil components are triglycerides bearing fatty acid residues. It has proven particularly advantageous, especially in terms of physiological tolerability, if the oil component comprises a medium-chain triglyceride (MCT). Preferably, the medium-chain triglyceride has a structure that is a glycerol esterified with fatty acids having from 6 to 14 carbon atoms. More preferably, the medium-chain triglyceride is a glycerol esterified with fatty acids consisting of at least 90 mole percent caprylic acid (C₈) and capric acid (C₁₀).

It is further preferred to employ vegetable oils as the oil component. It is particularly suitable for the oil-in-water emulsions of the present invention if the vegetable oils are selected from the group consisting of soybean oil and safflower oil or mixtures thereof. Another suitable oil is triisostearin.

It has also been found that mixtures of different oils also provide benefits. Thus, for example, the mixture of medium-chain triglyceride and additional vegetable oil, especially soybean oil and/or safflower oil, is particularly well tolerated physiologically and shows an optimum range of activity. In a preferred embodiment, the weight ratio of medium-chain triglyceride to vegetable oil is from 1:10 to 10:1, preferably from 5:1 to 1:5, more preferably from 2:1 to 1:2, particularly preferably from 1.5:1 to 1:1.5, and especially at 1:1.

The oil-in-water emulsion usually comprises the oil component in an amount of from 1 to 30% by weight, preferably from 2 to 20% by weight, particularly from 4 to 15% by weight, and especially from 5 to 10% by weight, respectively based on the total weight of the emulsion.

The oil-in-water emulsions may comprise at least one phospholipid as further components.

Phospholipids are phosphorus-containing amphiphilic lipids. In the organism, they participate as membrane lipids in the composition of the lipid bilayer of a biomembrane. They are composed of a hydrophilic head and two hydrophobic hydrocarbon tails.

Preferably, the oil-in-water emulsion contains a phospholipid that is usually selected from the group consisting of phosphatidylcholine, phosphatidylethanolamine, phosphatidylserine, phosphatidylinositol and sphingomyelin, as well as any mixtures thereof.

More preferably, the emulsion contains a lecithin, which is derived, in particular, from soybeans and/or eggs.

Lecithin is the classical name for a group of chemical compounds, the so-called phosphatidylcholines. These are lipids, more precisely phospholipids, which are composed of fatty acids, glycerol, phosphoric acid and choline. Lecithins are components of the cell membranes of animal and plant organisms.

In a particularly preferred embodiment, the emulsion comprises lecithin esterified with natural fatty acids. The fatty acids of the lecithin may be hydrogenated to increase stability against oxidative spoilage. Thus, for example, hydrogenated soy lecithin, which is commercially available under the designation of Emulmetik 320 (Lucas Meyer Cosmetic S.A.), may be used.

Preferably, the oil-in-water emulsions usually contain the phospholipids in an amount of from 0.1 to 5% by weight, preferably from 0.2 to 2.5% by weight, more preferably from 0.3 to 1.5% by weight, and especially from 0.4 to 1.2% by weight, respectively based on the total weight of the emulsion.

Especially with respect to the reduced cytotoxicity, it has proven advantageous to match the weight ratio of the antimicrobially active substance to the phospholipid. In a preferred embodiment, the weight ratio of the antimicrobially active substance to the phospholipid is from 1:4 to 1:40, preferably from 1:6 to 1:20, especially from 1:8 to 1:12.

Especially in view of the formation of suitable oil-in-water emulsions that meet the conditions for a low cytotoxicity, it is further advantageous if the weight ratio of the antimicrobially active substance to the oil component is from 1:80 to 1:1000, preferably from 1:100 to 1:800, more preferably from 1:150 to 1:600, especially from 1:180 to 1:450.

It has proven particularly advantageous if the wound dressings according to the invention have a composition containing the above described oil-in-water emulsion and, as the antimicrobially active substance, components having at least one guanide and/or biguanide group.

The antimicrobially active substance is usually contained in an amount of up to a maximum of 10% by weight, particularly from 0.25 to 5% by weight, and especially in an amount of from 0.3 to 4% by weight, based on the cytotoxicity-reducing composition.

Further, it has surprisingly been found that the particle size of the emulsified oil droplets is advantageously to be adjusted in a nanoscale range. In a preferred embodiment of the present invention, the emulsified oil droplets have a mean particle size of up to 300 nm, preferably from 30 to 260 nm, more preferably from 50 to 100 nm. The determination of the particle size is effected by photon correlation spectroscopy (Autosizer II Malvern Instruments UK) at 20° C.

The oil-in-water emulsion additionally contains one or more alkanediols and/or one or more glycerol ethers. The use of alkanediols in the cytotoxicity-reducing composition is particularly preferred.

The use of alkanediols may promote wound healing and even contribute to an increase of effectiveness against particular germs, such as yeasts (Candida). In addition, it has been found that when alkanediols are used in the oil-in-water emulsions, the concentration of the antimicrobially active substances can be reduced and the antimicrobial effect maintained as compared to emulsions without alkanediols. This additionally leads to a further reduction of cytotoxicity.

It has been found that suitable alkanediols include those having from 3 to 12 carbon atoms, in particular. Particularly preferred alkanediols are selected from the group consisting of 1,2-propylene glycol, 1,3-butylene glycol, 1,2-pentanediol, 1,2-hexanediol, 1,6-hexanediol and/or 1,2-octanediol. 1,2-Alkanediols having from 5 to 10 carbon atoms are preferred. 1,2-Octanediol is particularly preferred.

The alkanediols are preferably employed in amounts of from 0.01 to 10% by weight, especially from 0.05 to 5% by weight, respectively based on the total weight of the polymeric substrate and the composition.

Glycerol ethers may also be preferably contained in the oil-in-water emulsions. They exhibit a cytotoxicity-reducing effect, especially when acting together with the oil-in-water emulsion.

The components set forth below correspond to INCI nomenclature.

The glycerol ethers are preferably selected from the group consisting of octoxyglycerol, polyglyceryl-3-methylglucose distearate, polyglyceryl-6 polyhydroxystearate, polyglyceryl laurate, and polyglyceryl caprate.

Preferred glycerol ethers also include the glycerol monoalkyl ethers, such as 1-(2-ethylhexyl) glycerol ether.

The glycerol ethers are preferably employed in amounts of from 0.01 to 10% by weight, especially from 0.1 to 5% by weight, respectively based on the total weight of the polymeric substrate and the composition.

### i) Betaine Derivatives

As additional ingredients, the composition may contain one or more betaine derivatives having a surface-active effect as the cytotoxicity-reducing agent. In addition to the favorable effect reducing the cytotoxicity of the antimicrobial substances, the betaine derivatives additionally contribute advantageously to the softening and dissolution of the wound debris, to the removal of biofilms, and to the moisturizing of the wound. In addition, the betaine derivatives additionally make an anti-inflammatory contribution. Among the group of betaine derivatives, the alkylbetaines and alkylamidopropylbetaines, especially undecylene-amidopropylbetaine (chemical name: (carboxymethyl)dimethyl[3-[(1-oxoundecenyl)amino]propyl]ammonium hydroxide), but also cocoamidopropylbetaine, capryl/capramidopropylbetaine, ricinolamidopropylbetaine, laurylbetaine as well as lauryldimethylbetaine have proven particularly advantageous.

The betaine derivatives are preferably employed in amounts of from 0.01 to 10% by weight, especially from 0.05 to 5% by weight, respectively based on the total weight of the polymeric substrate and the composition.

### ii) Fatty Alcohol Alkoxylates

The following fatty alcohol alkoxylates are named according to the INCI nomenclature, if it deviates from the IUPAC nomenclature.

As additional ingredients, fatty alcohol alkoxylates may be contained in the composition as a cytotoxicity-reducing agents. Preferably, the fatty alcohol alkoxylates are C₁₂-C₁₈ fatty alcohols etherified with on average from 2 to 15 ethylene oxide and/or propylene oxide units. Suitable examples include laureth-3 (C₁₂ fatty alcohol ethoxylate 3 ED), laureth-7 (C₁₂ fatty alcohol ethoxylate 7 ED), laureth-9 (C₁₂ fatty alcohol ethoxylate 9 EO), Ceteareth-25 (C_{16/18} fatty alcohol ethoxylate 25 EO), O_{12/14} fatty alcohol ethoxylate 2 EO+4 PO, C_{12/14} fatty alcohol ethoxylate 3 EO+6 PO, C_{12/14} fatty alcohol ethoxylate 5 EO+4 PO, ceteareth-60 myristyl glycol C₁₆₋₁₈ alkyl polyethylene glycol tetradecylene glycol ether, PPG-3 stearyl ether (stearyl alcohol 3 PO), and PPG-15 stearyl ether (stearyl alcohol 15 PO).

The “ED” and “PO” groups represent the number of ethylene glycol ether groups (EO) or the number of propylene glycol ether groups (PO).

The fatty alcohol alkoxylates are preferably employed in amounts of from 0.01 to 10% by weight, especially from 0.05 to 5% by weight, respectively based on the total weight of the polymeric substrate and the composition.

iii) Glycerol Esters

The components set forth below correspond to INCI nomenclature.

As additional ingredients, glycerol esters may be contained in the composition as a cytotoxicity-reducing agent.

Preferably, the glycerol esters are selected from the group consisting of glycerol fatty acid esters, especially glyceryl caprate and glyceryl laurate.

The glycerol esters are preferably employed in amounts of from 0.01 to 10% by weight, especially from 0.1 to 5% by weight, respectively based on the total weight of the polymeric substrate and the composition.

### iv) Allantoin

In addition, allantoin may be employed in the composition as a cytotoxicity-reducing agent. It has surprisingly been found that allantoin not only reduces the cytotoxicity of the antimicrobially active substances, but additionally accelerates cell reproduction and cell regeneration, and thus supports wound healing. Allantoin (chemical name: N-(2,5-dioxo-4-imidazolidinyl)urea) is the end product, in addition to uric acid, of the degradation of nucleic acids, especially purine bases, in various animal species, mainly in mammals. In addition, allantoin is a substance contained in some indigenous plants, especially in comfrey. In addition, allantoin can be found in salsify, but also in wheat germs, soybean germs, rice, cauliflower, green beans and horse chestnut. Allantoin is preferably employed in amounts of from 0.05 to 2% by weight, especially from 0.1 to 1% by weight, respectively based on the total weight of the polymeric substrate and the composition.

### v) Panthenol or Pantothenic Acid

As additional ingredients, panthenol and/or pantothenic acid may be employed in the composition as a cytotoxicity-reducing agent. Panthenol additionally supports the reproduction of skin cells and moreover has an anti-inflammatory effect. Panthenol is also referred to as dexpanthenol and is converted to pantothenic acid in the body. Pantothenic acid is a vitamin from the group of B vitamins (vitamin B5).

Panthenol and/or pantothenic acid are preferably employed in an amount of from 0.05 to 5% by weight, more preferably from 0.1 to 2% by weight, especially from 0.2 to 1% by weight, respectively based on the total weight of the polymeric substrate and the composition.

In addition, the composition may contain further components that can promote wound healing and/or cell reproduction or reduce cytotoxicity. Polyalkylene glycols have proven particularly suitable for this purpose. Therefore, the composition may contain polyalkylene glycols as additional ingredients.

Polyalkylene glycols (polyglycols, polyglycol ether; INCI Chemical Class: Polymeric Ethers) are known polyethers, which are predominantly linear, but in part also branched, which are polymers having terminal hydroxy groups. The polyalkylene glycols having higher molecular weights are polymolecular, i.e., they consist of statistical universes of macromolecules having different molecular weights.

The average relative molecular weights of the polyalkylene glycols are preferably within a range of from 200 to 10,000, especially from 500 to 8,000, more preferably from 1000 to 6000, and most preferably from 1500 to 5000. In the different polyalkylene glycols described below, particular ranges may be still particularly advantageous.

Linear or branched, especially linear, polyalkylene glycols of general formula HO—[R—O]ₙ—H are preferred according to the invention, wherein R represents (CH₂)₂, CH₂CH(CH₃), CH₂CH(CH₂CH₃) and/or (CH₂)₄, and n represents values or averages of from 2 to about 200, preferably from 3 to 190, more preferably from 4 to 180, particularly preferably from 6 to 150, especially from 10 to 120. The polyalkylene glycols may be prepared by ring-opening polymerization of ethylene oxide, propylene oxide and/or tetrahydrofuran. These are, in particular, the polyethylene glycols with R═(CH₂)₂, the polypropylene glycols with R═CH₂CH(CH₃), the polytetrahydrofurans with R═(CH₂)₄, and the copolymers of ethylene oxide, propylene oxide and/or tetrahydrofuran.

Polyethylene glycols (PEGs) having an average relative molecular weight of from 400 to 10,000 are preferred according to the invention, especially from 1,000 to 8,000, more preferably from 2,000 to 6,000, and most preferably from 3,000 to 5,000. There are different nomenclatures for polyethylene glycols, which may lead to confusion. In technology, it is common to put the average relative molecular weight after the letters “PEG”, so that “PEG 200” characterizes a polyethylene glycol having a relative molecular weight of about 190 to about 210. According to the INCI nomenclature, the symbol PEG is provided with a hyphen directly followed by a number that corresponds to the number n in the above general formula. Commercially available polyethylene glycols include, for example, PEG 200/PEG-4, PEG 300/PEG-6, PEG-7, PEG-8, PEG 400, PEG-9, PEG-10, PEG-12, PEG 600, PEG-14, PEG-16, PEG 800/PEG-18, PEG-20, PEG 1000, PEG 1200, PEG 1500/PEG-32, PEG-40, PEG 2000, PEG-55, PEG-60, PEG 3000, PEG 3350/PEG-75 and PEG 4000/PEG-90, wherein the designations according to the two nomenclatures for mutually corresponding polyethylene glycols are juxtaposed, separated by the sign “/”. The commercially available polyethylene glycols are available, for example, under the trade names Carbowax® (Union Carbide), Emkapol® and Renex® PEG (ICI), Lipoxol® (DEA), Polyglykol® E (Dow), Pluracol® E, Pluriol® E and Lutrol® E (BASF).

Polypropylene glycols (PPG) are clear, almost colorless liquids over a broad range of molecular weights from 250 (PPG-4) to 4,000 (PPG-69), for the designation of which the above described INCI nomenclature is used similarly. Thus, the polypropylene glycols of the above general formula with values n of 5 and 6 are referred to as PEG-5 and PEG-6, respectively. The low molecular weight polypropylene glycols are miscible with water, while the higher molecular weight representatives are less soluble in water. For example, the polypropylene glycols PPG-7, PPG-9, PPG-12, PPG-13, PPG-15, PPG-17, PPG-20, PPG-26, PPG-30, PPG-33, PPG-34, PPG-51 and PPG-69 as designated according to INCI are commercially available. Sources of supply can be seen from the International Cosmetic Ingredient Dictionary and Handbook.

The copolymers are preferably random copolymers and, in particular, block copolymers of ethylene oxide and propylene oxide, ethylene oxide and tetrahydrofuran, propylene oxide and tetrahydrofuran, or ethylene oxide, propylene oxide and tetrahydrofuran, preferably of ethylene oxide and propylene oxide, more preferably block copolymers of ethylene oxide and propylene oxide.

Random copolymers formed from “a” ethylene oxide moieties and “b” propylene oxide moieties that are preferred according to the invention include, for example, the following copolymers designated according to the International Cosmetic Ingredient Dictionary and Handbook as PEG/PPG-a/b (molecular weight), wherein a and b represent mean values: PEG/PPG-18/4 copolymer (1000), PEG/PPG-17/6 copolymer (1100), PEG/PPG-35/9 copolymer (2100) and PEG/PPG-23/50 copolymer (3900).

Block copolymers of ethylene oxide and propylene oxide that are preferred according to the invention meet the formula HO(CH₂CH₂O)ₓ(CH(CH₃)CH₂O)_{y}(CH₂CH₂O)_{x′}H, in which x and x′ represent mean values from 2 to 130, and y represents mean values from 15 to 67, and are designated with the international non-proprietary name “poloxamer”, which is also used in the International Cosmetic Ingredient Dictionary and Handbook. Each poloxamer is identified by a three-digit number. The first two digits when multiplied by 100 represent the average molecular weight of the polypropylene glycol fraction, and the last digit multiplied by 10 represents the polyethylene glycol fraction in percent by weight. The latter is from 10 to 80% by weight, preferably not more than 50% by weight, especially not more than 40% by weight, more preferably not more than 30% by weight, for example, 10, 20 or 30% by weight. The production of the poloxamers is effected in two stages, wherein propylene oxide is first added to propylene glycol in a controlled way, and the polypropylene glycol block obtained is flanked by two polyethylene glycol blocks by the subsequent addition of ethylene oxide. Particularly preferred block copolymers include, for example, the following liquid poloxamer types (x, y, x′; molecular weight; in part melting point): poloxamer 101 (2, 16, 2; 1100; −32), poloxamer 122 (5, 21, 5; 1630; −26), poloxamer 123 (7, 21, 7; 1900; −1), poloxamer 105 (11, 16, 11; 1850; 7), poloxamer 181 (3, 30, 3; 2000; −29), poloxamer 124 (11, 21, 11; 2200; 16), poloxamer 182 (8, 30, 8; 2500; −4), poloxamer 183 (10, 30, 10; 2650; 10), poloxamer 212 (8, 35, 8; 2750; −7), poloxamer 231 (6, 39, 6; 2750; −37), poloxamer 184 (13, 30, 13; 2900; 16), poloxamer 185 (19, 30, 19; 3400), poloxamer 282 (10, 47, 10; 3650; 7), poloxamer 331 (7, 54, 7; 3800; −23), poloxamer 234 (22, 39, 22; 4200; 18), poloxamer 401 (6, 67, 6; 4400; 5), poloxamer 284 (21, 47, 21; 4600) and poloxamer 402 (13, 67, 13; 5000; 20). The poloxamers are commercially available under the trade names Pluronic® and Synperonic® PE, followed by a letter from the group L, P and F and a two- or three-digit number. The last digit is identical with the last digit of the poloxamer nomenclature, and the one- or two-digit numbers preceding it when multiplied by 300 yield the approximate molecular weight of the polypropylene glycol fraction, or when multiplied by 3, approximately yield the number formed by the first two digits of the poloxamer nomenclature number, i.e., 3, 4, 6, 7, 8, 9, 10 and 12 correspond, respectively, to the two-digit numbers 10, 12, 18, 21, 23, 28, 33 and 40 at the beginning of the number according to the poloxamer nomenclature. The letters distinguish between liquid (L), paste-like (P) and solid (F) poloxamers. Thus, for example, poloxamer 101 is obtainable as Pluronic® L 31 and Synperonic® PE L 31.

Another class of suitable block copolymers of ethylene oxide and propylene oxide correspond to the formula HO(CH(CH₃)CH₂O)_{y}(CH₂CH₂O)ₓ(CH₂CH(CH₃)O)_{y′}H. Here, one polyethylene glycol block is framed by two polypropylene glycol blocks, while one polypropylene glycol block is flanked by two polyethylene glycol blocks in the poloxamers. The production is again effected in two steps, wherein ethylene oxide is first added to ethylene glycol in a controlled way, and the polyethylene glycol block obtained is flanked by two polypropylene glycol blocks by the subsequent addition of propylene oxide. Like the poloxamers, these block copolymers are commercially available under the trade name Pluronic® (BASF), each followed by an alphanumeric code of three digits and the letter R inserted between the second and third digits. The meaning of the digits is the same as the meaning within the poloxamer nomenclature. The inserted letter R (for reverse) indicates the inverted structure as compared to the poloxamers. Preferred representatives of this class include the following Pluronic® types (molecular weight; melting point): Pluronic® 10R5 (1950; 15), Pluronic® 12R3 (1800; −20), Pluronic® 17R1 (1900; −27), Pluronic® 17R2 (2150; −25), Pluronic® 17R4 (2650; 18), Pluronic®25R1 (2700; −5), Pluronic® 25R2 (3100; −5), Pluronic® 31R1 (3250; −25) and Pluronic® 31R2 (3300; 9).

In another preferred embodiment, one or more of the terminal hydroxy groups of the above mentioned alkylene glycols may be additionally etherified. In the preferred terminal etherifled alkylene glycols, the hydrogen atoms of the hydroxy groups are substituted by linear or branched, saturated or unsaturated alkyl groups with 1 to 30 carbon atoms.

The polyalkylene glycols are advantageously employed in an amount of from 0.001 to 8% by weight, preferably from 0.01 to 5% by weight, more preferably from 0.01 to 3% by weight, especially from 0.01 to 1.5% by weight, respectively based on the total weight of the polymeric substrate and the composition.

Preferably, the weight ratio of polymeric substrate to composition is from 1:5 to 1:30, preferably from 1:10 to 1:20, in the case where the composition is contacted with a polymeric substrate, i.e., applied to the substrate.

In the case where the composition is present during the polymerization of the polymeric substrate, the weight ratio of prepolymer mix to composition is from 80:1 to 0.1:1, more preferably from 50:1 to 0.1:1.

The present invention further relates to the use of the wound dressing according to the invention for the treatment of wounds, especially wounds that are susceptible to microbiological contamination, or that are microbiologically contaminated or infected, especially burn injuries, diabetes and decubitus ulcer.

In a preferred embodiment, the wound dressing according to the invention may also have a multilayer design. Preferably, for example, the polymeric substrate of the wound dressing according to the invention is coated with an adhesive on one side thereof, which ensures a good wound contact, but prevents the wound dressing from agglutinating with the wound, especially when the uptake of wound exudate is low. This adhesive layer is preferably made of silicone. In another preferred embodiment, the wound dressing is covered by a moisture-impermeable layer, for example, a polymer film, on the side facing away from the wound, in order to facilitate the handling of the wound dressing. Such a polymer film may preferably be made of silicone, polyamide or a water-impermeable polyurethane.

The present invention further relates to a process for preparing a wound dressing, wherein a polymeric substrate is prepared by a polymerization reaction in the presence of a composition comprising
a) at least one antimicrobially active substance; and
b) a cytotoxicity-reducing agent comprising an oil-in-water emulsion that additionally contains one or more alkanediols and/or one or more glycerol ethers.

In a particularly preferred embodiment, the polymerization reaction for preparing the polymeric substrate is a reaction of a polyisocyanate with a polyol.

The present invention further relates to a process for preparing a wound dressing according to the present invention, comprising the following steps:
a) providing a prepolymer mix for the preparation of a polyurethane foam;
b) providing an aqueous composition comprising
   1) at least one antimicrobially active substance; and
   2) a cytotoxicity-reducing agent comprising an oil-in-water emulsion that additionally contains one or more alkanediols and/or one or more glycerol ethers; and
c) combining the prepolymer mix and the aqueous composition to form a polyurethane foam; and
d) optionally sterilizing, preferably by gamma rays or by means of high-pressure steam sterilization.

Suitable and preferred prepolymers that may be employed for the process according to the invention have been described above. The present invention further relates to a process for preparing a wound dressing according to the invention, comprising the following steps:
a) providing a liquid-absorbing polymeric substrate; and
b) contacting the substrate with an aqueous composition comprising
   1) at least one antimicrobially active substance; and
   2) a cytotoxicity-reducing agent comprising an oil-in-water emulsion that additionally contains one or more alkanediols and/or one or more glycerol ethers; and
c) optionally drying; and
d) optionally sterilizing, preferably by gamma rays or by means of high-pressure steam sterilization.

Suitable and preferred components that may be contained in the compositions and that are employed in the processes according to the invention have been described above in connection with the wound dressing according to the invention.

### EXAMPLES

A liquid-absorbing hydrophilic polyurethane foam based on a prepolymer capped with toluene diisocyanate was prepared. The compositions used for preparing the foam are stated in Tables 1 and 2.

The prepolymer mix can be combined with the compositions described in Tables 1 and 2 in a suitable ratio, and the mixture can be foamed to achieve a desired final concentration of the antimicrobially active substance PHMB.

In all Examples, it is to be taken into account that part of the water may evaporate because of the preparation conditions.

The compositions set forth in the following Tables 1 and 2 show suitable compositions that may be applied to a polymeric substrate for the wound dressings according to the invention, or may be processed into a foam together with a suitable prepolymer.

TABLE 1
Oil-in-water emulsion with alkanediol to be employed according to the
invention

| Amount in weight percent (% by weight) | Component |
|---|---|
| 10% by weight | medium-chain triglyceride¹⁾ |
| 10% by weight | soybean oil |
| 2.25% by weight | 1,2-octanediol |
| 1.2% by weight | egg lecithin |
| 0.25% by weight | sodium oleate |
| 0.2% by weight | α-tocopherol |
| 1.5% by weight | PHMB (20%)²⁾ |
| ad 100 | water |

¹⁾Glycerol esterified with fatty acids consisting of up to 98 mole percent of caprylic acid (C₈) and capric acid (C₁₀)
²⁾Aqueous solution containing 20% by weight polyhexamethylene biguanide hydrochloride (PHMB)

TABLE 2
Oil-in-water emulsion with alkanediol to be employed according to the
invention

| Amount in weight percent (% by weight) | Component |
|---|---|
| 20% by weight | triisostearine |
| 2.25% by weight | 1,2-octanediol |
| 1.2% by weight | hydrogenated soy lecithin |
| 0.25% by weight | sodium isostearate |
| 1.5% by weight | PHMB (20%)¹⁾ |
| ad 100 | water |

¹⁾Aqueous solution containing 20% by weight polyhexamethylene biguanide hydrochloride (PHMB)

Surprisingly, it has been found that the use of aliphatic alkanediols, such as the 1,2-octanediol employed in Tables 1 and 2, enables a reduction of the antimicrobially active substance to be employed while the antimicrobial effect is the same. This further reduces cytotoxicity, because a lower concentration of the antimicrobially active substance is required for the same antimicrobial effect.

1. A wound dressing comprising a polymeric substrate and a composition comprising
a) at least one antimicrobially active substance; and
b) a cytotoxicity-reducing agent comprising an oil-in-water emulsion that additionally contains one or more alkanediols and/or one or more glycerol ethers.

2. The wound dressing according to claim 1, wherein said polymeric substrate can absorb liquid,

3. The wound dressing according to claim 1, wherein said polymeric substrate is selected from the group consisting of polyurethanes, polyethers, cellulose materials, cellulose derivatives, polyesters, polyvinyl alcohol, polyvinyl acetate, polysulfones, polyacrylates, polyolefins, polyamides, alginates, chitosan as well as mixtures and combinations thereof.

4. The wound dressing according to claim 1, wherein said polymeric substrate is selected from the group consisting of polyethylene terephthalate, polyethylene, polypropylene, rayon, cotton, nylon-6,6, polycaprolactam, polyurethane as well as any mixtures and combinations thereof.

5. The wound dressing according to claim 1, wherein said polymeric substrate is polyurethane.

6. The wound dressing according to claim 5, wherein said polyurethane is obtained by reacting a component having at least one isocyanate group with at least one polyol.

7. The wound dressing according to claim 1, wherein said antimicrobially active substance is selected from the group of components having at least one guanide and/or biguanide group, iodophors, triclosan, octenidine, quaternary ammonium compounds, lactoferrin, undecylenic acid and/or salts thereof and/or any mixtures thereof.

8. The wound dressing according to claim 1, wherein said antimicrobially active substance is selected from the group consisting of polymethylene biguanide, polyhexamethylene biguanide, polyhexamethylene guanide, chlorhexidine, hexetidine, iodophors, PVP-iodine, 2,4,4′-trichloro-2-hydroxydiphenyl ether, octenidine, and salts thereof.

9. The wound dressing according to claim 1, wherein said composition additionally contains one or more further cytotoxicity-reducing agents selected from the group consisting of
one or more betaine derivatives;
one or more fatty alcohol alkoxylates;
one or more glycerol esters;
allantoin; and
panthenol and/or pantothenic acid.

10. The wound dressing according to claim 1, wherein said antimicrobially active substance is obtainable by the polycondensation of a guanidine acid addition salt with a mixture of amines which contains at least one diamine and/or triamine.

11. The wound dressing according to claim 10, wherein said guanidine acid addition salt is guanidine hydrochloride, and/or said mixture of amines comprises an alkylene diamine.

12. The wound dressing according to claim 10, wherein said mixture of amines comprises diethylene triamine, and/or said mixture of amines comprises 4,4′-methylenebis(cyclohexylamine).

13. The wound dressing according to claim 1, wherein said antimicrobially active substance has an average relative molecular weight within a range of from 500 to 7000.

14. The wound dressing according to claim 1, wherein said antimicrobially active substance is present in an amount of up to a maximum of 10% by weight, based on the total weight of the polymeric substrate and the composition.

15. The wound dressing according to claim 1, wherein said oil-in-water emulsion comprises at least one phospholipid and at least one oil component selected from medium-chain triglycerides, soybean oil, safflower oil, isostearin, or mixtures thereof.

16. The wound dressing according to claim 1, wherein said composition contains one or more betaine derivatives.

17. The wound dressing according to claim 1, wherein said composition contains a component selected from the group consisting of fatty alcohol alkoxylates.

18. The wound dressing according to claim 1, wherein said composition contains pantothenic acid and/or dexpanthenol.

19. The wound dressing according to claim 1, wherein said composition contains allantoin.

20. The wound dressing according to claim 1, wherein said composition contains one or more polyalkylene glycols with an average molecular weight of from 200 to 10,000 daltons.

21. The wound dressing according to claim 1, wherein said composition contains one or more alkanediols having from 3 to 12 carbon atoms.

22. The wound dressing according to claim 21, wherein said alkanediol is 1,2-octanediol.

23. The wound dressing according to claim 1, wherein said composition contains one or more glycerol ethers and/or glycerol esters.

24. A method for treating a wound, said method comprising using the wound dressing according to claim 1.

25. A process for preparing a wound dressing, wherein a polymeric substrate is prepared by a polymerization reaction in the presence of a composition comprising
a) at least one antimicrobially active substance; and
b) a cytotoxicity-reducing agent comprising an oil-in-water emulsion that additionally contains one or more alkanediols and/or one or more glycerol ethers.

26. The process according to claim 25, wherein the polymerization of the polymeric substrate is effected by a reaction of a polyisocyanate with a polyol.

27. A process for preparing a wound dressing according to claim 1, comprising the following steps:
a) providing a prepolymer mix for the preparation of a polyurethane foam;
b) providing an aqueous composition comprising
1) at least one antimicrobially active substance; and
2) a cytotoxicity-reducing agent comprising an oil-in-water emulsion that additionally contains one or more alkanediols and/or one or more glycerol ethers; and
c) combining the prepolymer mix and the aqueous composition to form a polyurethane foam; and
d) optionally sterilizing.

28. A process for preparing a wound dressing according to claim 1, comprising the following steps:
a) providing a liquid-absorbing polymeric substrate; and
b) contacting the substrate with an aqueous composition comprising
1) at least one antimicrobially active substance; and
2) a cytotoxicity-reducing agent comprising an oil-in-water emulsion that additionally contains one or more alkanediols and/or one or more glycerol ethers; and
c) optionally drying; and
d) optionally sterilizing.
